(19) United States
(12) Patent Application Publication
(10) Pub. No.: US 2017/0166662 A1
(43) Pub. Date: Jun. 15, 2017

(54) HIGH PURITY CELLULOSE COMPOSITIONS AND PRODUCTION METHODS

(71) Applicant: Glucan BioRenewables, LLC, St. Louis, MO (US)

(72) Inventors: David Martin Alonso, Madison, WI (US); Jeffrey J. Fornero, Colinsville, IL (US); Sikander Hakim, Madison, WI (US)

(73) Assignee: Glucan BioRenewables, LLC, St. Louis, MO (US)

(21) Appl. No.: 15/216,363

(22) Filed: Jul. 21, 2016

Related U.S. Application Data
(60) Provisional application No. 62/195,454, filed on Jul. 22, 2015.

(51) Int. Cl. C08B 1/00; B01D 21/00; B01D 21/26; C07D 307/50

(52) U.S. Cl. CPC C08B 1/00; C07D 307/50; B01D 21/0012; B01D 21/262

(57) ABSTRACT

The present disclosure relates generally to the field of biomass refining and cellulose production. More particularly, it concerns high purity cellulose production using a process that employs a water/organic co-solvent mixture with reduced use of chemicals.

This application claims benefit of priority to U.S. Provisional Application Ser. No. 62/195,454, filed Jul. 22, 2015, the entire contents of which are hereby incorporated by reference.

### BACKGROUND

1. Field

The present disclosure relates generally to the field of biomass refining and cellulose production. More particularly, it concerns high purity cellulose production in a process that employs a water/organic co-solvent mixture with reduced use of chemicals.

2. Description of Related Art

Biomass is considered as the most promising renewable alternative to petroleum-based chemicals and fuels. Biomass is abundant and widely spread in the world. Biomass is comprised of three main components, cellulose, hemicellulose, and lignin. Cellulose is a crystalline polymer comprised of β-D-glucopyranose units linked via β-glycosidic bonds, hemicellulose is an amorphous polymer comprised of five and six carbon sugars with β 1,4 linkages, and lignin is an amorphous polymer composed of methoxylated phenylpropane structures. Currently, biomass conversion processes to produce chemicals are focused on the depolymerization of the cellulose and hemicellulose to produce monomeric sugars that can be used as platform molecules to produce many chemicals and biofuels by fermentation or catalytic upgrading. The use of the cellulose polymer as a raw material is limited to few applications such as in the pulp and paper industry due to the high capital and operation cost required to produce a cellulose stream with desired purity. New technologies producing higher value cellulose products such as viscose pulp and fiber, cellulose nanocrystals (CNC), and nanocellulose (cellulose nanofibrils, CNF) are promising, but these require a much higher purity of cellulose, which is difficult to achieve while keeping production costs low. Additionally, the methods to increase the cellulose purity result in property degradation of the hemicellulose and lignin. In this regard, a widespread utilization of cellulose will require cost-effective methods to produce high purity solid cellulose while retaining the value of the hemicellulose and the lignin fractions.

### SUMMARY

Disclosed herein is a method for producing a high purity cellulose stream without degrading other components present in the cellulose source. The method comprises (a) one or more sources of cellulose, (b) treating said source with a recirculated solvent, comprised of water, an acid and one or more aprotic organic solvents, (c) avoiding re-precipitation of dissolved material in the recirculated solvent onto the cellulose, (d) partially separating the liquid fraction from the solid cellulose, (e) washing the solid cellulose at conditions to prevent the re-precipitation of dissolved material, and (f) recovering the organic solvents used in the process.

The source of cellulose can be lignocellulosic biomass, such as corn stover, corn cobs, sugarcane bagasse, hardwood, softwood, palm oil empty fruit bunches, paper pulp, paper sludge, municipal solid waste residue. The biomass may be present in a concentration range selected from the group consisting of from about 5 wt % to about 70 wt %, from about 5 wt % to about 50 wt %, from about 10 wt % to about 50 wt %, from about 10 wt % to about 30 wt %, from about 15 wt % to about 35 wt %, from about 15 wt % to about 30 wt %, based on the total weight of the biomass and solvent system. Low biomass loadings may produce better results and higher purity cellulose; however they are not economically viable because of the associated costs required for the recovery and purification of the product and solvent. In this aspect, working at a higher biomass loading (>15%) is preferred. The concentration of the products can be increased by successive biomass additions during the process. Thus, the biomass may be reacted in a single batch or by multiple additions in a semi-batch operation or can be added continuously. At high biomass concentrations the selection of the solvents is critical to achieve desired cellulose purity.

The aprotic organic solvent can be any aprotic organic solvent but in particular it is produced from biomass, preferentially within the process or in one additional step and it is capable of solubilizing high concentrations of lignin, biomass derived degradation products and water. With these requirements, the organic solvent may be a lactone, a lactam an ether, a furan, an alcohol, an organic acid, or combinations thereof, for example γ-valerolactone, butyrolactone, hexalactone, pyrrolidone, methyl pyrrolidone, tetrahydrofuran (THF), furan, methyl tetrahydrofuran (MTHF), dioxane, levulinic acid, formic acid, acetic acid, or sulfolane and more specifically may be γ-valerolactone (GVL). The water can be directly added to the mixture or can enter as part of the cellulose stream (for example, wet biomass). To facilitate the solubilization of sugars derived from the partial degradation of the cellulose or hemicellulose (if present), it is recommended that 5-30% water is present. The amount of water present in the solvent can be increased to 60% in some reaction conditions depending on the biomass type and loading and final target of cellulose purity. When water or wet biomass is mixed with the recirculated solvent, precipitation of dissolved materials within the solvent must be avoided. This can be achieved by reducing or increasing the amount of water (depending on the solubility of the dissolved material in water and in the solvent) or by treating the solvent prior to recirculation to remove dissolved impurities.

The acid may be a homogeneous acid, a heterogeneous acid, a Brønsted-Lowry acid, a Lewis Acid, a solid acid, a mineral acid, an organic acid, or any combination of these. (Note that any given acid might be described by more than one of the foregoing identifiers.) If homogeneous, the acid is present in dilute concentration, in particular no greater than about 1000 mM. Thus, acid concentrations between about 0.1 mM and about 500 mM are particularly contemplated, more particularly between about 5 mM and about 500 mM, and more particularly still between about 5 mM and about 250 mM. On a weight percentage basis, based on the weight of the lactone/water solvent, the acid is particularly present in an amount of about 0.001 wt % to about 5.0 wt %, more particularly from about 0.01 wt % to about 0.25 wt %.

The biomass and the solvent system may be reacted at a temperature from about 50° C. to about 250° C. and for a time from about 1 minute to about 24 hours. The temperature may be held constant or the biomass and the solvent system may be reacted at a dynamic temperature range. For example, the dynamic temperature range may include an optional temperature ramp from a first temperature to a second temperature that is higher or lower than the first temperature. The temperature ramp may be linear, non-linear, discontinuous, or any combination thereof.

Treatment time may vary at the choice of the user, and be adjusted empirically based on the selection of the cellulose source. Generally, though, it is preferred that the solvent have a residence time in the reactor of from 1 min to 24 hours. Residence times above and below these extremes are within the scope of the process. Thus, the process explicitly covers residence times selected from the group consisting of 1 min to 24 hours, 1 min to 20 hours, 1 min to 12 hours, 1 min to 6 hours, 1 min to 3 hours, 1 min to 2 hours, 1 min to 1 hour, and 1 min to 30 min.

The separation of the liquid from the solid can be performed by any method described in the literature, such as, centrifugation, decantation, filtration, or compression. In any case, 100% liquid removal is not necessary, but the liquid has to be separated from the solid at conditions to prevent the re-precipitation of the dissolved material. This can be achieved by performing the separation at the reaction temperature and/or minimizing the evaporation of the organic solvent to prevent supersaturation. The solvents (such as GVL) with high boiling points compared with other biomass-derived solvents presents an advantage.

The removal of remaining liquid with the solid cellulose can be done by washing at conditions to prevent re-precipitation of dissolved materials onto the solid cellulose. The same solvent or a different solvent can be used for the washing. The solvent used for the washing can be removed by evaporation, but it is preferred to remove the solvent by a water washing to minimize the precipitation of dissolved solids. The removal of the solvent does not need to be complete and some solvent could remain with the final cellulose.

The liquid separated from the cellulose can be treated to remove the soluble species. Ideally these species are removed in a way that retains their value so that they can be upgraded to high value chemicals. For example, dissolved hemicellulose can be converted into furfural, while lignin can be burned to generate heat or used to produce chemicals or bioproducts. Dissolved products can be removed from the solvent by precipitation of the products, evaporation of the products, evaporation of the solvent, or combinations thereof. The solvent can be recovered in different steps and in different streams. These streams may or may not be mixed and proportions may vary depending on the composition. Complete cleaning of the solvent before recirculation is not necessary and it is preferred that the recirculated solvent has dissolved material in it when entering in the reactor in order to build concentrations.

In yet another embodiment, there is provided a method of producing a nano-crystalline cellulose composition comprising (a) providing lignocellulosic biomass; (b) treating said lignocellulosic biomass with a mineral acid and an aprotic solvent that preferentially solubilizes lignocellulosic biomass materials other than cellulose; (c) generating a liquid stream of the materials in step (b); and (d) separating solid nano-crystalline cellulose from the liquid stream of step (c). The method may further comprise purifying cellulose before producing nano-crystalline cellulose. The cellulose may be treated with concentrated acid to remove the non-crystalline cellulose prior to separating the nano-crystalline cellulose. The non-nano-crystalline cellulosic portion of the cellulose may be further treated to produce a distinct chemical substance within the solvent fraction, such as where the chemical substance is glucose, HMF, levulinic acid, GVL or derivatives thereof.

In still a further embodiment, there is provided a composition comprising high purity solid cellulose with low hemicellulose and low lignin content, and retaining native cellulose properties, wherein said solid cellulose comprises no more than 2% w/w of GVL. The native properties may comprise two or more of crystallinity, strength, fiber length, and viscosity. The solid cellulose may have an alpha cellulose content of 60%, 90%, or 90-99%, or any range derivable therefrom. The low hemicellulose content may be no more than about 3%, no more than about 2% hemicellulose, no more than about 1.5% hemicellulose, no more than about 1.0% hemicellulose, or no more than about 0.5% hemicellulose. Low lignin content comprises less than about 2% lignin, no more than about 1.5% lignin, no more than about 1.0% lignin, or no more than about 0.5% lignin. The obtained cellulose may have a viscosity average molecular weight in the range 100,000 to 200,000. The obtained cellulose may have uniform molecular weight. The obtained cellulose may be dissolving grade. The obtained cellulose may have a purity of 60%, 70%, 80%, 90%, 95%, or 90%-98%.

When using biomass as the cellulose source with the mixed solvent medium, the reaction time, temperature and the number and quantity of the chemicals required for the solubilization of the hemicellulose and lignin components is lower as compared to when using pure aqueous medium. Recommended values of temperature range is from about 100° C. to about 150° C., or more precisely 100° C. to about 140° C. Using a lower temperature is advantageous to prevent degradation of soluble sugars and the production of carbon residues, humins. The temperature can be adjusted during the reaction to optimize the solubilization of fractions other than cellulose and to prevent the solubilization of the cellulose while the production of some dehydration products is unavoidable; the properties of the solvents allow process conditions to minimize this degradation reaction.

The feedstock particle size (e.g., wood chip size) can be adjusted to have an effect on final pulp properties. The smaller particle sizes are advantageous for higher cellulose yield and lower kappa number, while the larger particles sizes are advantageous for the pulp viscosity.

Since the pulp has a starting lower kappa number and higher brightness as compared to in traditional pulping processes, the bleaching sequences can be modified to achieve higher brightness for paper and viscose production such that viscosity is not compromised.

Embodiments discussed in the context of methods and/or compositions of the disclosure may be employed with respect to any other method or composition described herein. Thus, an embodiment pertaining to one method or composition may be applied to other methods and compositions of the disclosure as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The U.S. patent or application file contains at least one drawing executed in color. Copies of the U.S. patent or patent application publication with color drawing(s) will be provided by the office by request and payment of the necessary fees.

FIG. 1 is a flow chart depicting a method to treat a source of cellulose to produce a stream of high purity solid cellulose. The solvent is at least partially recovered and recycled.

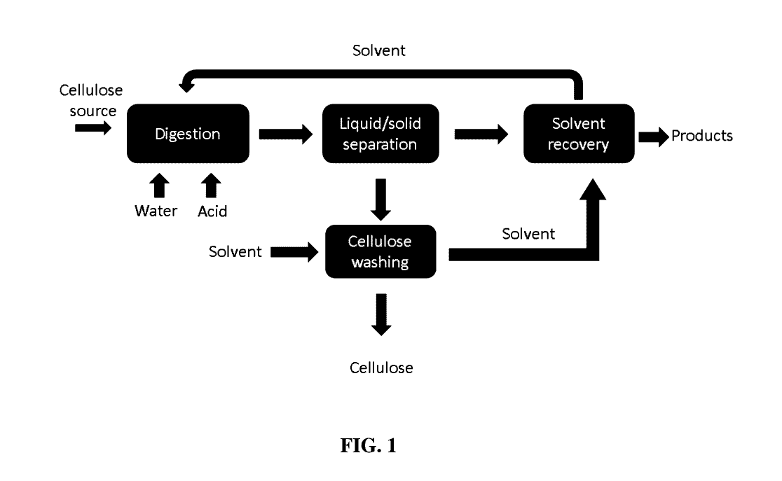

FIG. 2 presents a flow chart depicting a method to fractionate lignocellulose biomass. The biomass is digested to separate the hemicellulose and lignin from the cellulose. The cellulose is washed and after recovering at least part of the solvent, a solid stream of high purity cellulose is produced. The solvent can be recycled to process more lignocellulosic biomass. The hemicellulose and the lignin can be upgraded to products after the separation of the cellulose. The solvent with the soluble hemicellulose and lignin is at least partially recovered. Depending on the solvent purity, the solvent can be used to wash the cellulose before recycling it to treat more lignocellulosic biomass.

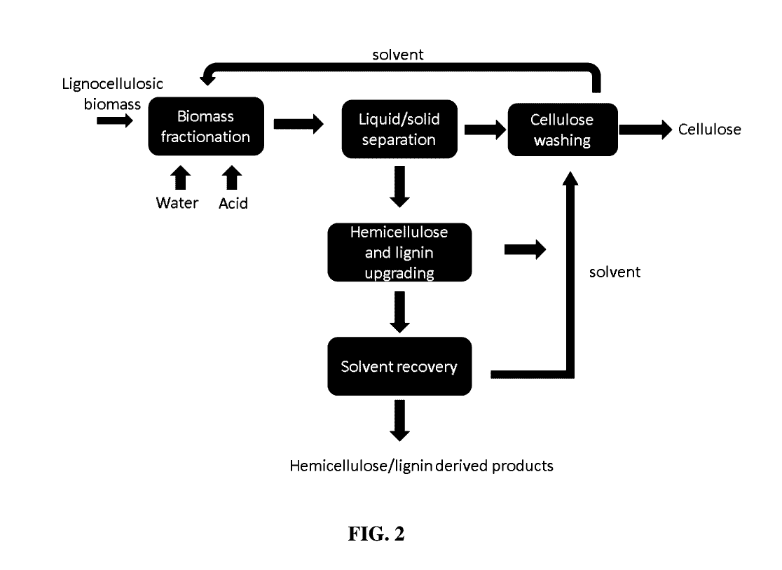

FIG. 3 is a histogram showing the extraction of hemicellulose for 18 wt % white birch wood chips at 140° C. using as solvent 80/20 wt % GVL/water solution with 0.1 M sulfuric acid. Samples were retrieved at various time intervals. The maximum hemicellulose extraction is achieved between 30 and 45 min. Increasing the reaction duration only increased the amount of furfural produced. An efficient washing step is necessary to remove all the hemicellulose from the solid cellulose. Less than 10% of the cellulose is extracted as soluble sugars or dehydration products.

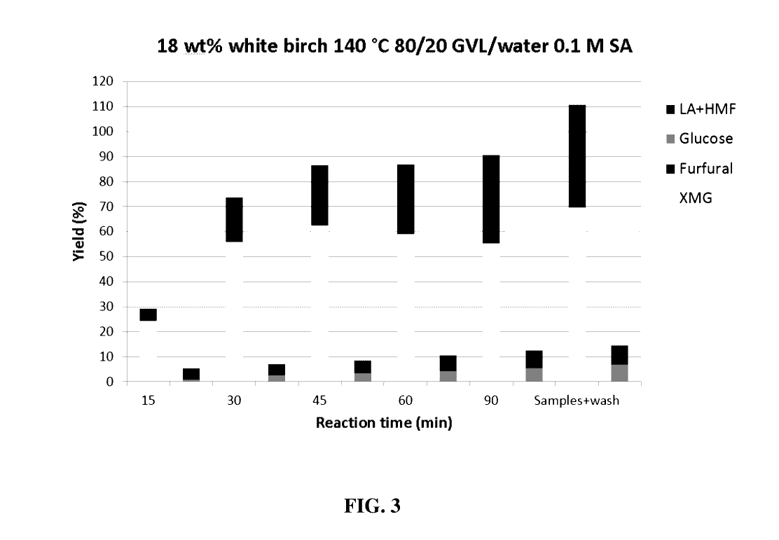

FIG. 4 is a histogram showing the extraction of hemicellulose for 15% white birch wood chips at 125° C. using 70/30 w/w GVL/water as a solvent mixture and 0.1 M sulfuric acid. The analysis was performed on the liquor obtained at the completion of reaction after a duration of 3 hours. An efficient washing step was necessary to remove all hemicellulose from within the solid cellulose. Less than 10% of cellulose was extracted as soluble sugars or dehydration products.

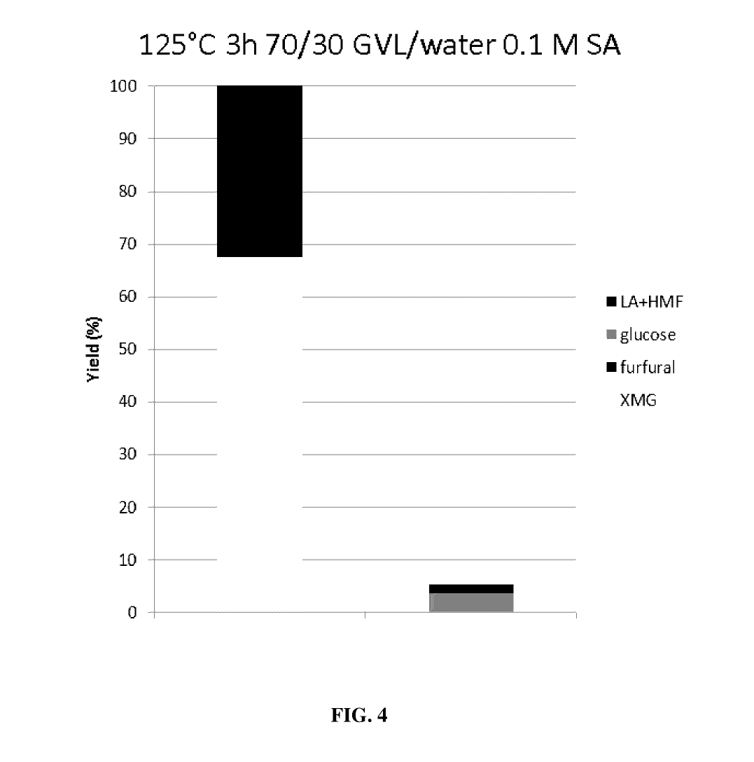

FIG. 5 is a histogram showing the extraction of hemicellulose for 10 wt % shredded palm oil empty fruit bunches (POEFB) at 130° C. using as solvent 80/20 wt % GVL/water solution with 0.075 M sulfuric acid. Samples were taken at various time intervals. The maximum hemicellulose extraction is achieved between 30 and 45 min. Increasing the reaction duration only increased the amount of furfural produced. Less than 10% of the cellulose is extracted as soluble sugars or dehydration products. Longer reaction times increased the amount of cellulose hydrolyzed.

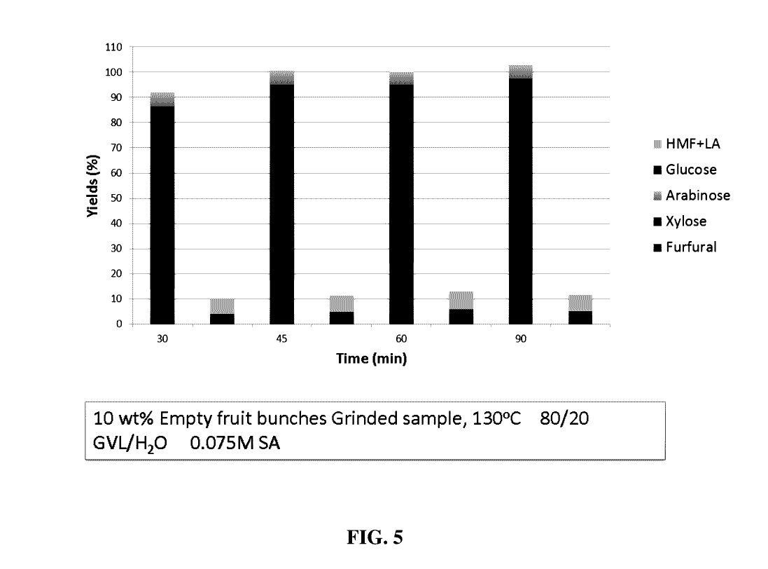

FIG. 6 is a histogram showing the extraction of hemicellulose for 10 wt % grinded white birch wood at 130° C. using as solvent 80/20 wt % GVL/water solution with 0.1 M H₂SO₃. The maximum hemicellulose extraction is achieved between 60 and 120 min. Less than 10% of the cellulose is extracted as soluble sugars or dehydration products.

FIG. 7 is a histogram showing the composition of white birch and the solids recovered after treating white birch chips with 80/20 wt % GVL/water solution with 0.1 M H₂SO₄. An effective washing step with a solvent is necessary to achieve high purity cellulose without further chemical processing.

### DESCRIPTION OF ILLUSTRATIVE EMBODIMENTS

### I. INTRODUCTION

The present disclosure addresses the production of high purity solid cellulose from one or more sources of cellulose. The method comprises reacting the cellulose sources with a solvent system comprising water, at least an acid, and at least an organic aprotic solvent, for a time and at a temperature to yield a solid fraction (cellulose) and a liquid fraction enriched with other materials contained in the cellulose source, for example, in the case of processing lignocellulosic biomass the liquid would be enriched in lignin, extractives and/or hemicellulose (monomeric sugars, oligomeric sugars and/or dehydration products and/or degradation products). The liquid and solid fractions can then be separated for post-treatment upgrading of one or both fractions. An important part of the innovation is that the solvent is recirculated after separation of part of the dissolved material and used again in the process. Thus, the solvent may contain soluble impurities. Solvent selection and process conditions are chosen to avoid re-precipitation over the cellulose. This step is critical when high concentrations of biomass are used in the process. The separation of the cellulose and the liquid is done in such a way that even though part of the solvent is still present with the cellulose after the liquid-solid separation, the material solubilized remains soluble. A washing step is included to remove the remaining liquid within the cellulose after the initial solid-liquid separation without precipitation of soluble material onto the cellulose. This washing step and solvent selection is necessary to obtain high purity cellulose without further chemical processing. If the concentration of soluble species is low enough, the remaining liquid can be removed from the cellulose by evaporation. All the solvents used in the process are at least partially recovered from the cellulose and reutilized in the process. The cellulose will contain small amount of the solvents used in the process.

The initial step is treating the cellulose source with a liquid solvent to dissolve the hemicellulose, extractives, lignin, and other materials present while retaining the cellulose as a solid. The reaction can be done by any known method. Many solvents and reactor configurations have been proposed in the literature. Most treatments have been done using water at low or high pH, also many organic solvents have been proposed to facilitate the lignin removal (Nissan, 1984, Wyman at <world-wide-web at wiley.com/WileyCDA/WileyTitle/productCd-0470972025.html>; Xu and Huang, 2014).

PCT/US2014/070963 describes a method to produce a high concentration solution of C₅ sugars and a solid cellulose stream from lignocellulosic biomass. The method describes a process to produce a liquid stream enriched in C₅ sugars and a solid cellulose stream using an organic solvent. The method can extract more than 95% of the C₅ sugars present in lignocellulosic biomass and also can solubilize part of the lignin.

During the digestion process, C₅ and some C₆ sugars are released into the liquid media. While these released sugars can be separated from the cellulose by a water wash, it has been reported that sugars in the presence of organic solvents can easily be dehydrated to products, including degradation products and humins that remain soluble in the organic solvent and represent a risk to re-precipitate onto the cellulose, thus decreasing its purity (Gallo et al., 2013, Gürbüz et al., 2013 and Alonso et al., 2012). Also, the presence of the organic solvent may increase the rate of cellulose hydrolysis, converting it into glucose (Mellmer et al., 2014). Although, these side reactions are not critical for producing cellulose, they are important to understand and manage as they may affect the high purity cellulose yield. Therefore, the reaction conditions must be carefully controlled on a case by case basis, depending on cellulose source type, loading, etc. The main process control parameters are the digestion temperature, reaction duration, and acid concentration. These three variables have been extensively studied in the biomass pretreatment literature and several factors have been proposed to evaluate the severity of the treatment, for example, a combined severity factor or a combined hydrolysis factor (Lee and Jeffries, 2011 and Zhu et al., 2012). The preferred combined severity factor range from 0.5 to 2.5 even though the disclosure is not limited to that range. At lower combined severity factors, not all of the hemicellulose may be removed from the cellulose resulting in a lower cellulose purity. At higher severity factors, degradation products re-precipitate over the cellulose surface and cellulose is hydrolyzed to glucose, which reduces the cellulose purity and yield. Besides above three variables, another important parameter effecting the cellulose properties and yields is the feedstock particle size (e.g., wood chip size).

In the digestion process, at least one homogeneous or heterogeneous is used. The acid may be a mineral acid, an organic acid, etc. The acid may be present in the solvent system in a concentration sufficient to yield a [H⁺] concentration selected from the group consisting of about 0.005M to about 0.5M, about 0.05M to about 0.3M, about 0.05 to about 0.25M. Concentrations above and below these ranges are, however, within the scope of the method. Because the hydrolysis reaction rates are greater in the organic solvents than in pure water, the amount of acid necessary to perform the reaction is lower than the amount of acids required in water-based processes. This reduces the use of additional chemicals during the process.

The ratio of the organic solvent to water has an effect on the hemicellulose and lignin extraction, which in turn affects the purity of the final cellulose. In all reactions described herein, the ratio of organic solvent-to-water is preferably at least about 60 wt % organic solvent (or higher) to about 40 wt % water (or lower) (60:40; organic solvent:water). Thus, explicitly included within the disclosed process are ratios of organic-to-water of 60:40, 65:35, 70:30, 75:25, 80:20, 85:15, 90:10, 95:5, 97:3, 98:2, 99:1, and any ratio that falls between the two extremes. Preferably the organic solvent is miscible with water, or can dissolve from 2 wt % to 40 wt % water. The method can be conducted using γ-valerolactone (GVL) as the organic solvent. As noted above, the organic solvent may be present in a ratio with water (organic solvent:water) selected from the group consisting of about 60:40, about 65:35, about 70:30, about 75:25, about 80:20, about 85:15, about 90:10, about 95:5, about 97:3, about 98:2, and about 99:1. The source of any water present may be from the biomass itself, added to the solvent prior to digestion, or added after Typically, the choice of the organic solvent-water ratio has been chosen based on the reaction performance (Fang and Sixta, 2015 and Nguyen et al., 2015). In the present method, at least part of the solvent is recirculated and may contain some dissolved impurities that may re-precipitate over the cellulose if the solvent is supersaturated. This occurs mostly when high loadings of biomass are used, as is the case in the disclosure. Precipitation of impurities or other materials will reduce the purity of the cellulose making the further treatment necessary in order to obtain purities >90%. For this reason, one of the deciding factors regarding the choice of the organic solvent-water ratio is the solubility of the compounds other than cellulose present in the cellulose source and in the recycled solvent. Another factor dictating the choice of organic solvent—water ratio is the influence of final cellulose properties and in that will be influenced by the properties being targeted.

The effect of the reaction temperature is included in the severity factor. Temperatures from 100° C. to 250° C. are possible depending on the reaction time and acid concentration, but for cellulose purity and yield considerations, temperatures between 100° C. and about 150° C., or more precisely 100° C. to about 140° C. are particularly contemplated. The possibility of using low temperatures is advantageous versus other processes because the resultant process pressure (due to the presence of water) in the reactor will be lower, facilitating the reactor loading and downstream operations. It is well known that hemicellulose and lignin are removed following two parallel first order reactions with one of them much faster than the other (Zhu et al., 2012 and Mittal et al., 2015). The kinetics are modified in presence of the organic solvents (Mellmer et al., 2014 and Mellmer et al., 2014b) but the two phase kinetics remain. Better results are possible by starting the reaction at a specific temperature and then decreasing or increasing the temperature during the reaction. Indeed, more than one temperature change is beneficial in some cases to maximize the purity of the cellulose and its mechanical and chemical properties.

The digestion hydraulic retention time of the biomass and solvent in the reactor may vary at the choice of the user, and be adjusted empirically based on the selection of the biomass, biomass-derived reactant, temperature, and acid concentration. Generally, though, it is preferred that the solvent have a residence time in the reactor from 1 min to about 120 min, or 1 min to about 180 min, but the reaction time could be increased up to 24 h depending on the choice of temperature, acid concentration and acid strength.

The treatment of the cellulose source with the solvent can be performed in a batch or in a continuous operation. In one version of the method, the cellulose source and the liquid solvent are loaded into the reactor separately. The amount of water in the liquid solvent is adjusted depending on the moisture content of the cellulose source. At the end of the reaction, the solid cellulose is separated from the liquid by taking out the liquid from the reactor. The cellulose, still containing part of the solvent, will remain in the reactor for further washing and purification. The washing step is done with the same solvent that was used to treat the cellulose in the first place. If the concentration of soluble products is far from the saturation point, the solvent used to wash the cellulose is used to treat more cellulose. Alternatively, if the concentration of soluble products is close to the saturation point, the solvent is treated to remove part of the soluble material. In another version, both cellulose and solvent are taken out of the reactor and separated using, for example a centrifuge.

In another version of the method, a continuous reactor is used. The cellulose source and solvent are loaded continuously into a reactor and processed at the desired reaction conditions. At the end of the reaction the solid cellulose is separated from the liquid and both streams are processed separately.

In another version of the method, the biomass is introduced into in the reactor and the solvent mixture with acid is sprayed to wet the biomass using an atomizing spray nozzle.

Any other method or reaction configuration can be used to treat the cellulose source.

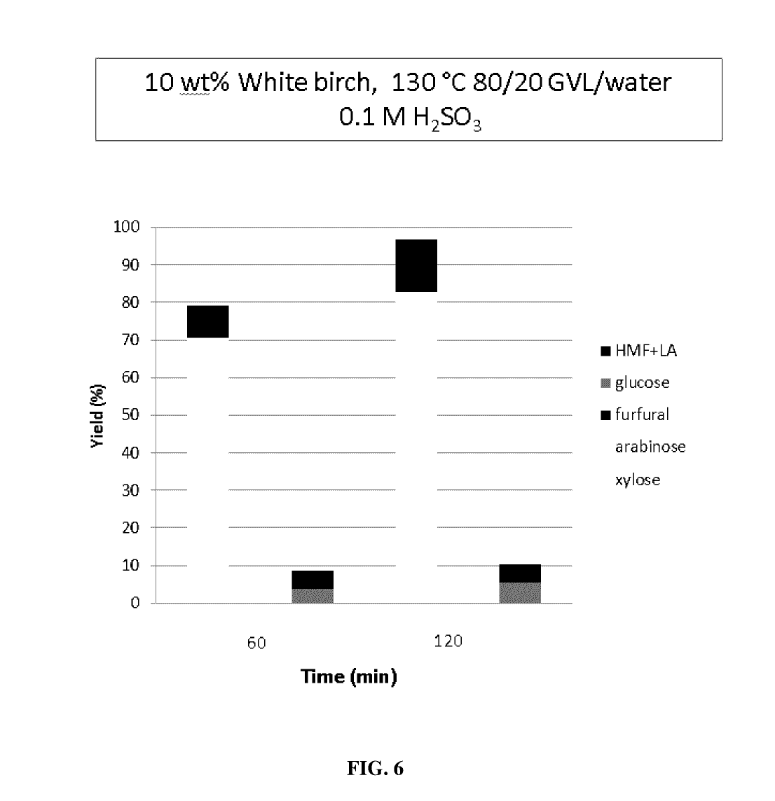

Any solid-liquid separation method will yield a solid stream of wetted cellulose. The liquid retained in the cellulose may still contain soluble material that can precipitate onto the cellulose upon removal of the solvent. Even though the operation of solvent removal appears to be obvious, the special characteristics of the solvents used, the variety of different soluble materials present in the solvents (sugars, lignin, extractives, degradation product, reaction byproduct, salts, ash, etc.), the difference in solubilities of these materials in water, organic solvents and mixtures thereof, and the pH dependent solubility of many of those materials makes the successful production of high purity cellulose challenging. Many experts in the field have treated lignocellulosic biomass to remove lignin and hemicellulose, however, they have encountered significant limitations when attempting to produce high purity cellulose, mainly due to insufficient solubilization of the lignin and/or hemicellulose during the reaction. For example, Sixta and cowokers (Fang and Sixta, 2015) obtained excellent results to extract lignin and hemicellulose from white birch. Using 1 g of grinded white birch, 10 g of 50% water/50% GVL and 0.05 M sulfuric acid as solvent at 150° C. for 45 minutes, yielded a 0.4 g solid residue (40.05% referred to the initial biomass), which contained 0.345 g of glucan (34.5% of the initial material). These results indicate a cellulose purity of 86%. Lignin, glucose, xylose and other sugars only yielded 0.38 g (38.12% of the initial material) indicating that other impurities (ash, degradation products, etc.) also reduced the cellulose purity and that these also have to be considered. A simple modification of the reaction conditions is not sufficient to improve the results as the authors obtained even lower purities at other reaction conditions. The importance of the solid-liquid separation and washing step is even more significant if one considers that the authors removed extractives before the reaction using acetone. Another example of treating biomass with an organic solvent and water is the work by Wyman group (Nguyen et al., 2015) who tried to produce a cellulose stream suitable for enzymatic hydrolysis by removing the hemicellulose and the lignin. Using 5 wt % corn stover biomass, THF as solvent (1:1 ratio) with 0.5 wt % sulfuric acid, and treatment at 150° C. for 25 minutes, the authors were only able to produce a cellulose stream with 75% purity, even though the cellulose showed excellent results for enzymatic conversion. In one scenario in the current work in the inventors' lab, treating 18 wt % white birch with 80/20 GVL water by weight and 0.1 M sulfuric acid at 130-140° C. for 45-60 minutes removed >90% of the hemicellulose and >90% of the lignin to yield a 75-80% cellulose purity with a conventional washing step after the reaction (FIG. 6). When the liquid is only partially separated from the solid cellulose and washed at the appropriate conditions, using a mixture of water and GVL at different proportions during the washing step, the purity of the cellulose increased to >91% without requiring the use of additional treatments such as bleaching or caustic washing.

It is preferred that the solvent selected to wash the cellulose is able to solubilize all the materials present in the cellulose, other than cellulose. In general a combination of solvents is preferred, for example, water can be used to remove water-soluble impurities, while organic solvents can be used to remove organic impurities. Organic solvents with different polarities can be used in the process, but it is preferred if the same solvent that was used to treat the cellulose is used. When using a combination of solvents, they can be used separately or mixed together. When mixed, the proportions of the solvents can be changed during the washing procedure.

The cellulose has to be treated to recover the organic solvents used in the process. The recovery of the solvent has to be done at conditions to prevent re-precipitation of the dissolved material. For example, removal of the solvent by evaporation can be utilized, but only after the dissolved non-cellulose material, or at least most of it, has been removed from the cellulose. Removal of 100% of the solvent is not necessary in this step and the cellulose will retain some of the solvent. In particular cases where the presence of the solvent has a negative effect on the cellulose properties or down-stream processing, the remaining solvent can be substantially removed to leave a very small residual quantity with the cellulose.

The solvent used to treat the cellulose source has to be recovered and recirculated. The recovery of this solvent can be done before or after separating soluble materials such as hemicellulose or lignin. Hemicellulose, for example, can be processed in the solvent and used to produce furfural, (Gürbüz et al., 2013, Mellmer et al., 2014b; Gallo et al., 2013) then the furfural can be separated from the solvent and used as a product. The lignin can be separated before or after producing furfural and be used as fuel or to produce chemicals and/or bioproducts (Zakzeski et al., 2010).

Optionally part or the totality of the solvent can be cleaned to remove the soluble impurities before recycling the solvent. This can be done by precipitating the soluble impurities, evaporating the solvent, combinations thereof or any other method known or to be developed to clean the solvent.

In some cases the addition or the presence of acid can help during the washing. In this case special care has to be taken into consideration to prevent further degradation of the cellulose, mostly by hydrolysis to produce glucose and soluble oligomers. The high purity of the cellulose without further chemical treatment makes it an excellent feedstock to be used for enzymatic hydrolysis, chemical production, viscose pulp and fiber production, nano-crystalline cellulose, pulp and paper applications, cellulose nanofibers (CNF), and in general any application that requires a high cellulose purity.

The special characteristics of the solvent and mild process conditions have an effect of the mechanical properties. Even though the degree of polymerization is reduced due to the action of the acid, the cellulose retains enough strength to produce paper pulp compared with cellulose prepared by other methods and similar degree of polymerization.

The effective removal of hemicellulose, lignin and extractives by the effect of GVL/water during biomass digestion is advantageous in utilization of the final high purity cellulose in the production of viscose pulp and fiber because it has very low hemicellulose content, non-detectable to zero levels of ash, acid insoluble and lignin which is desirable for viscose production.

Fast cellulose hydrolysis within aprotic solvents is an effective pre-treatment to achieve high cellulose purity and facilitate the production of nano-cellulose. Lignin, hemicellulose, amorphous cellulose and other biomass components can be easily removed at mild conditions being an option to reduce the energy cost of the overall process to produce nano-cellulose and can even facilitate the mechanical disintegration of the cellulose. When using an acid hydrolysis as a pretreatment method for the nano-cellulose production, the faster hydrolysis rates can results in shorter processing time, lower temperatures and lower acid concentration, all of them affecting to the final properties of the nano-cellulose. An important parameter of the process is the flocculation of the nano-cellulose that is different in the aprotic solvents and the water.

When the target is the production of nano-crystalline cellulose, the use of aprotic solvents modifies the crystallinity of the cellulose, which at the appropriate process conditions can lead to increased yields. Using lower acid concentrations and milder process conditions can also affect the crystallinity and lead to more advantageous process conditions improving the final yields. Recovery and utilization of by products such as removed amorphous cellulose, glucose and/or dehydration products produced during the process can be converted into levulinic acid and this, hydrogenated into GVL with no or minimal separations and improving the overall carbon utilization within the process.

Because the cellulose has an extraordinary purity, one or more of its mechanical properties is enhance compared with the cellulose produced by other methods. The improved properties enable a better performance in some applications and open the possibility of using the cellulose in new applications. Some of the properties considered are viscosity, strength, surface area, chemical resistance, crystallinity, particle size, and accessibility.

Some applications may require a higher purity or other physical and mechanical characteristic that can be achieve by the described method. In these cases, the cellulose produced by the method described herein can be treated with a base or bleached by any method already known or to be developed. There are many examples of bleaching sequences in the literature (Nissan, 1984). GVL cooked cellulose offers an advantage due to a more effective delignification and extraction that results in higher starting brightness and lower kappa number (lower oxidative demand) thus requiring lower number and quantities of chemicals in bleaching process.

Another version of the method includes reacting biomass with a recycled organic solvent (GVL or THF or mixtures) that was previously used to fractionate biomass. The organic solvent may or may not contain acid and water when recycled. If water and acid are present, they may not be in the correct proportions required for the reaction. More than one organic solvent may be present in the solution, if used in another unit operation of the process, as well as other organic compounds derived from the biomass conversion. Inorganic materials present in the biomass or added to the system during neutralization streams may be present as well. The solvent may also contain soluble degradation products and/or soluble lignin if those have not been completely removed in the process during a solvent purification step. The water content in the solvent may be adjusted to about 70:30, about 75:25, about 80:20, about 85:15, about 90:10, and about 95:5. The acid content in the solvent may be adjusted to about 0.05M to about 0.5M, about 0.05M to about 0.5M, about 0.05 to about 0.2M, and about 0.05 to about 0.15M. The biomass is treated, for a time from about 1 min to about 120 min or 1 min to about 180 min, and at a temperature from about 100° C. to about 150° C., or more precisely 100° C. to about 140° C. wherein the reaction yields a liquid fraction and a solid fraction enriched in substantially insoluble cellulose. The liquid fraction is carefully separated from the solid fraction to prevent any precipitation of the dissolving material. Special care has to be taken to prevent evaporation of the solvent as this may cause the precipitation of the dissolved material by saturation of the liquid. While this is not a problem at low loading of biomass (<5%) as is typical of other methods, it has important considerations when higher biomass loading (>15 wt %) are used in the process as is the case disclosed herein. The biomass concentration can range from about 5 wt % to about 70 wt %, but it is preferably for the system from about 15 wt % to about 35 wt %, based on the total weight of the biomass and solvent system. The concentration of the products can be increased by successive additions of biomass during the process or recirculation of the solvent following the cellulose separation Thus, the biomass may be reacted in a single batch or in multiple additions or continuously.

### II. DEFINITIONS

A. Abbreviations and Chemical Definitions

“Severity factor” or “combined severity factor” is defined as a number to combine the effect of several reaction variables in a single parameter. As defined here, it combines the effect of temperature, acid concentration and reaction time following the equation in aqueous media:

$$CSF = log ⌊ t \times exp ( \frac{T - 100}{14.75} ) ⌋ - pH or$$

$$CSF = log ⌊ t \times exp ( \frac{T - 100}{14.75} ) ⌋ + log [ acid concentration ]$$

where t is the reaction time and T is the reaction temperature.

“Biomass” as used herein includes materials containing cellulose, hemicellulose, lignin, protein and carbohydrates such as starch and sugar. Common forms of biomass include trees, shrubs, crops and grasses, as well as municipal solid waste, waste paper and yard waste. Biomass high in starch, sugar or protein such as corn, grains, fruits and vegetables, is usually consumed as food. Conversely, biomass high in cellulose, hemicellulose and lignin is not readily digestible by humans and is primarily utilized for wood and paper products, fuel, or is discarded as waste. Biomass explicitly includes but not limited to branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines, hard and soft woods. In addition, biomass includes organic waste materials generated from agricultural processes including farming and forestry activities, specifically including forestry wood waste. “Biomass” includes virgin biomass and/or non-virgin biomass such as agricultural biomass, commercial organics, construction and demolition debris, municipal solid waste, waste paper, and yard waste. Municipal solid waste generally includes garbage, trash, rubbish, refuse and offal that is normally disposed of by the occupants of residential dwelling units and by business, industrial and commercial establishments, including but not limited to: paper and cardboard, plastics, food scraps, scrap wood, saw dust, and the like.

“Biomass-derived” means compounds or compositions fabricated or purified from biomass.

Cellulose source is any solid material that contains cellulose

A Bronsted-Lowry acid is defined herein as any chemical species (atom, ion, molecule, compound, complex, etc.), without limitation, that can donate or transfer one or more protons to another chemical species. Mono-protic, diprotic, and triprotic acids are explicitly included within the definition. A Bronsted-Lowry base is defined herein as any chemical species that can accept a proton from another chemical species. Included among Bronsted-Lowry acids are mineral acids, organic acids, heteropolyacids, solid acid catalysts, zeolites, etc. as defined herein. Note that this list is exemplary, not exclusive. The shortened term “Bronsted” is also used synonymously with “Bronsted-Lowry.”

“Carbohydrate” is defined herein as a compound that consists only of carbon, hydrogen, and oxygen atoms in their defined ratios.

“C₅ carbohydrate” refers to any carbohydrate, without limitation, that has five (5) carbon atoms. The definition includes pentose sugars of any description and stereoisomerism (e.g., D/L aldopentoses and D/L ketopentoses). C₅ carbohydrates include (by way of example and not limitation) arabinose, lyxose, ribose, ribulose, xylose, and xylulose.

“C₆ carbohydrate” refers to any carbohydrate, without limitation, that has six (6) carbon atoms. The definition includes hexose sugars of any description and stereoisomerism (e.g., D/L aldohexoses and D/L ketohexoses). C₆ carbohydrates include (by way of example and not limitation) allose, altrose, fructose, galactose, glucose, gulose, idose, mannose, psicose, sorbose, tagatose, and talose.

“Cellulose” refers to a polysaccharide of glucose monomers ((C₆H₁₀O₅)n); “cellulosic biomass” refers to biomass as described earlier that comprises cellulose, and/or consists essentially of cellulose, and/or consists entirely of cellulose. Lignocellulosic biomass refers to biomass comprising cellulose, hemicellulose, and lignin. Lignocellulosic biomass comprises xylose, as does hemicellulose.

“Nano-cellulose: is cellulosic material with one dimension in the nanometer range. This may be either cellulose nanofibers microfibrillated cellulose nanocrystalline cellulose. Nano-crystalline cellulose is a particular form of nano cellulose with high crystallinity.

Hemicellulose is the term used to denote non-cellulosic polysaccharides associated with cellulose in plant tissues. Hemicellulose frequently constitutes about 20-35% w/w of lignocellulosic materials, and the majority of hemicelluloses consist of polymers based on pentose (five-carbon) sugar units, such as D-xylose and D-arabinose units, and hexose (six-carbon) sugar units, such as D-glucose, D-mannose and D-galactose units. Generally, hardwood hemicellulose contains more xylose and softwood hemicellulose more mannose.

Lignin, which is a complex, cross-linked polymer based on variously substituted hydroxyphenylpropane units, generally constitutes about 10-30% w/w of lignocellulosic materials. It is believed that lignin functions as a physical barrier to the direct bioconversion (e.g., by cellulase) of cellulose and hemicellulose in lignocellulosic materials which have not been subjected to some kind of pre-treatment process (which may very suitably be the SPORL process as described in relation to the present disclosure) to disrupt the structure of lignocellulose.

The biomass material may be wood, such as hardwood and softwood, or herbaceous feedstock. Biomass refers to living and recently dead biological material that can be used as fuel or for industrial production. Most commonly, biomass refers to plant matter grown for use as biofuel, but it also includes plant or animal matter used for production of fibers, chemicals or heat. Biomass may also include biodegradable wastes that can be burnt as fuel. In certain embodiments, biomass may be grown crop fiber consisting primarily of cellulose, hemicellulose and lignin, and includes, without limitation, grass, switchgrass, straw, corn stover, cane residuals, general cereal wastes, wood chips and the like, that can be converted to ethanol (or other products) according to U.S. Pat. No. 4,461,648 and U.S. Pat. No. 5,916,780, or other known technology, incorporated herein by reference.

B. Hardwood

Hardwood comprises wood from broad-leaved (mostly deciduous, but not necessarily, in the case of tropical trees) or angiosperm trees. On average, hardwood is of higher density and hardness than softwood, but there is considerable variation in actual wood hardness in both groups, with a large amount of overlap; some hardwoods (e.g., balsa) are softer than most softwoods, while yew is an example of a hard softwood. Hardwoods may have broad leaves and enclosed nuts or seeds such as acorns. They may grow in subtropical regions like Africa and also in Europe and other regions such as Asia. The dominant feature separating hardwoods from softwoods is the presence of pores, or vessels. Examples of hardwood are described in U.S. Patent Publication 2009/0298149, incorporated herein by reference.

C. Softwood

Softwood is a generic term used in woodworking and the lumber industries for wood from conifers (needle-bearing trees from the order Pinales). Softwood-producing trees include pine, spruce, cedar, fir, larch, douglas-fir, hemlock, cypress, redwood and yew. Softwood is also known as Clarkwood, Madmanwood, or fuchwood. Examples of softwood are described in U.S. Patent Publication 2009/0298149 (incorporated herein by reference).

D. Biomass feedstock

Biomass feedstock comes in many different types, such as wood residues (including sawmill and paper mill discards), municipal paper waste, agricultural residues (including corn stover, straw, hull and sugarcane bagasse), and dedicated energy crops, which are mostly composed of fast growing tall, woody biomass.

Corn stover comprises leaves and stalks of maize (Zea mays ssp. mays L.) plants left in a field after harvest. It makes up about half of the yield of a crop and is similar to straw, the residue left in field after harvest of any cereal grain. It can be used as a fuel for bioenergy or as feedstock for bioproducts. Maize stover, together with other cellulosic biomass, provides about the potential 1.3 billion tons of raw materials per year that could produce future fuel in the next 50 years.

Useful sources of straw include in particular cereals (cereal grasses), i.e., gramineous plants which yield edible grain or seed. Straw from, for example, oat (Avena spp., such as A. saliva), barley (Hordeum spp., such as H. vulgare), wheat (Triticum spp., including T. durum), rye (Secal cereale), rice (Oryza spp.), millet (e.g., species of Digitaria, Panicum, Paspalum, Pennisetum or Setana), sorghum (Sorghum spp., including S. bicolor var. durra (also referred to as “durra”) and milo), buckwheat (Fagopyrum spp., such as F. esculentum) and maize (also referred to as corn (Zea mays), including sweetcorn) is well suited for treatment according to the process of the disclosure.

As employed herein, the term “hull” generally denotes the outer covering, rind, shell, pod or husk of any fruit or seed, but the term as employed herein also embraces, for example, the outer covering of an ear of maize. Relevant hulls include hulls selected among the following: hulls from oat (Avena spp., such as A. saliva), barley (Hordeum spp., such as H. vulgare), wheat (Triticum spp., including T. durum), rye (Secal cereale), rice (Oryza spp.), millet (e.g., species of Digiftaa, Panicum, Paspalum, Pennisetum or Setaria), sorghum (Sorghum spp., including S. bicolor var. durra and milo), buckwheat (Fagopyrum spp., such as F. esculentum), maize (also known as corn (Zea mays), including sweetcorn), corn cob, rape-seed (from Brassica spp., such as B. napus, B. napus subsp. rapifera or B. napus subsp. oleifera), cotton-seed (from Gossypium spp., such as G. heraceum), almond (Prunus dulcis, including both sweet and bitter almond) and sunflower seed (Helianthus spp., such as H. annuus), bagasse, palm oil empty fruit bunches, oil palm chips, oil palm stalks, oil palm kernel shells, oil palm mesocarp, coconut shells, coconut husks, sago palm, sago bark, sago second layer bark, sago pith, and nipah palm leaves.

Hulls of cereals, including not only those mentioned among the above, but also hulls of cereals other than those mentioned among the above, are generally of interest in the context of the disclosure, and particular hulls, such as oat hulls and barley hulls, belong to this category. In this connection it may be mentioned by way of example that oat hulls are often available in large quantities at low cost as a by-product of oat-processing procedures for the production of oatmeal, porridge oats, rolled oats and the like. Other types of hulls of relevance in relation to processes of the disclosure include, for example, palm shells, peanut shells, coconut shells, other types of nut shells, coconut husk or other tropical tree products.

It should be noted that the native physical form, bulk and/or dimensions of cellulosic materials such as wood, straw, hay and the like will generally necessitate, or at least make it desirable, to carry out size reduction of the material (e.g., by milling, abrading, grinding, crushing, chopping, chipping or the like) to some extent in order to obtain particles, pieces, fibers, strands, wafers, flakes or the like of material of sufficiently small size and/or sufficiently high surface area to mass ratio to enable degradation of the material to be performed satisfactorily. In the case of wood, material of suitable dimensions will often be available as a waste product in the form of sawdust, wood chips, wood flakes, twigs and the like from saw mills, forestry and other commercial sources.

In contrast, numerous types of hulls, e.g., cereal grain or seed hulls in general, including oat hulls as employed in the working examples reported herein, have in their native form sufficiently small dimensions and a sufficiently high surface area to mass ratio to enable them to be used directly, as cellulosic materials in a process according to the present disclosure

“Glucose-containing oligomers, glucose-containing polymers, Glucose-containing reactant, C₆-containing reactant” are any chemical species, having any type of intramolecular bond type that comprises glucose or other C₆ sugar unit. The definition explicitly includes glucose-containing disaccharides (such as, but not limited to, sucrose, lactose, maltose, trehalose, cellobiose, kojibiose, nigerose, isomaltose, β,β-trehalose, α,β-trehalose, sophorose, laminaribiose, gentiobiose, turanose, maltulose, palatinose, gentiobiulose, etc.), trisaccharides (such as, but not limited to, isomaltotriose, nigerotriose, maltotriose, maltotriulose, raffinose, etc.), and larger oligosaccharides and polysaccharides, as well as large and more complex glucose-containing polymers and carbohydrates and other polymers and carbohydrates containing C₆ sugar units, such as, but not limited to, starch, amylase, amylopectin, glycogen, cellulose, hemicelluloses (e.g., xyloglucan, glucomannan, etc.), lignocellulose, and the like. Linear, branched, and macrocyclic oligomers and polymers containing glucose, including those found in biomass, are explicitly included within the definition Likewise, “xylose-containing oligomers, xylose-containing polymers, xylose-containing reactant, C₅-containing reactant” are any chemical species, having any type of intramolecular bond type, that comprises a xylose or other C₅ sugar unit.

“Heteropolyacid” means a class of solid-phase acids exemplified by such species as H₄SiW₁₂O₄₀, H₃PW₁₂O₄₀, H₃₊ₓPMo₁₂₋ₓVₓO₄₀ and the like. Heteropolyacids are solid-phase acids having a well-defined local structure, the most common of which is the tungsten-based Keggin structure. The Keggin unit comprises a central PO₄ tetrahedron, surrounded by twelve WO₆ octahedra. The standard unit has a net (⁻³) charge, and thus requires three cations to satisfy electroneutrality. If the cations are protons, the material functions as a Bronsted acid. The acidity of these compounds (as well as other physical characteristics) can be “tuned” by substituting different metals in place of tungsten in the Keggin structure. See, for example, Bardin et al. (1998) “Acidity of Keggin-Type Heteropolycompounds Evaluated by Catalytic Probe Reactions, Sorption Microcalorimetry and Density Functional Quantum Chemical Calculations,” J. of Physical Chemistry B, 102:10817-10825.

“Homogeneous catalyst” means a catalyst that is soluble in the reaction solvent, and a “heterogeneous” catalyst is not soluble in the reaction solvent.

“Heterogeneous catalyst” means a catalyst that exists in a different phase than the reactants under reaction conditions.

“Lactone” as used herein refers to an unsubstituted or substituted cyclic ester, having a single oxygen heteroatom in the ring, and having from four to six total atoms in the ring, i.e., β-, γ-, and δ-lactones, derived from any corresponding C₄ to C₁₆ carboxylic acid. Thus, as used herein, the term “lactone” explicitly includes (without limitation) unsubstituted and substituted β- and γ-butyrolactone and β-, γ-, and δ-valerolactones to β-, γ-, and δ-hexadecalactones. Some lactones are miscible in water, such as GVL; other lactones have more limited solubility in water. Those lactones that can dissolve at least about 1 wt % water, and more preferably at least about 5 wt % (or more) of water (up to miscible) are suitable for use in the process described herein. γ- and δ-lactones are preferred. γ-valerolactone is most preferred.

Mineral acid is any mineral-containing acid, including (by way of example and not limitation), hydrochloric acid, nitric acid, phosphoric acid, sulfuric acid, boric acid, hydrofluoric acid, hydrobromic acid, and the like.

Organic acid is any organic acid, without limitation, such as toluenesulfonic acid, formic acid, acetic acid, trifluoroacetic acid, oxalic acid, and the like.

A Lewis acid is defined herein as any chemical species that is an electron-pair acceptor, i.e., any chemical species that is capable of receiving an electron pair, without limitation. A Lewis base is defined herein as any chemical species that is an electron-pair donor, that is, any chemical species that is capable of donating an electron pair, without limitation.

The Lewis acid (also referred to as the Lewis acid catalyst) may be any Lewis acid based on transition metals, lanthanoid metals, and metals from Group 4, 5, 13, 14 and 15 of the periodic table of the elements, including boron, aluminum, gallium, indium, titanium, zirconium, tin, vanadium, arsenic, antimony, bismuth, lanthanum, dysprosium, and ytterbium. One skilled in the art will recognize that some elements are better suited in the practice of the method. Illustrative examples include AlCl₃, (alkyl)AlCl₂, (C₂H₅)₂AlCl, (C₂H₅)₃Al₂Cl₃, BF₃, SnCl₄and TiCl₄.

“Sugars” are defined as short chain carbohydrates that are soluble in water.

The terms “solid acid” and “solid acid catalyst” are used synonymously herein and can comprise one or more solid acid materials. The solid acid catalyst can be used independently or alternatively can be utilized in combination with one or more mineral acid or other types of catalysts. Exemplary solid acid catalysts which can be utilized include, but are not limited to, heteropolyacids, acid resin-type catalysts, mesoporous silicas, acid clays, sulfated zirconia, molecular sieve materials, zeolites, and acidic material on a thermo-stable support. Where an acidic material is provided on a thermo-stable support, the thermo-stable support can include for example, one or more of silica, tin oxide, niobia, zirconia, titania, carbon, alpha-alumina, and the like. The oxides themselves (e.g., ZrO₂, SnO₂, TiO₂, etc.) which may optionally be doped with additional acid groups such as SO₄²— or SO³H may also be used as solid acid catalysts.

Further examples of solid acid catalysts include strongly acidic ion exchangers such as cross-linked polystyrene containing sulfonic acid groups. For example, the Amberlyst®-brand resins are functionalized styrene-divinylbenzene copolymers with different surface properties and porosities. These types of resins are designated herein as “Amb” resins, followed by a numeric identifier of the specific sub-type of resin where appropriate. The functional group is generally of the sulfonic acid type. The Amberlyst®-brand resins are supplied as gellular or macro-reticular spherical beads. Amberlyst® is a registered trademark of the Dow Chemical Co. Similarly, Nafion®-brand resins are sulfonated tetrafluoroethylene-based fluoropolymer-copolymers which are solid acid catalysts. Nafion® is a registered trademark of E.I. du Pont de Nemours & Co.

Solid catalysts can be in any shape or form now known or developed in the future, such as, but not limited to, granules, powder, beads, pills, pellets, flakes, cylinders, spheres, or other shapes.

Zeolites may also be used as solid acid catalysts. Of these, H-type zeolites are generally preferred, for example zeolites in the mordenite group or fine-pored zeolites such as zeolites X, Y and L, e.g., mordenite, erionite, chabazite, or faujasite. Also suitable are ultrastable zeolites in the faujasite group which have been dealuminated.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

Processes described herein include those run in batch mode, semi-continuous mode, and/or continuous mode, all of which are explicitly included herein.

E. End Products

The cellulose can be used for catalytic upgrading and produce fuels and chemicals as have been extensively reported (Dhepe and Fukuoka, 2008 and Van de Vyver et al., 2011). In particular the production of some derivatives, such as, levulinic acid, HMF or GVL can be an advantage if the solvent is present in the cellulose (Gallo et al., 2013, Alonso et al., 2012 and Wettstein et al., 2012)

The cellulose can be used for enzymatic hydrolysis and produce glucose. The high purity of the cellulose may have important advantages to reduce the amount of enzymes required for the hydrolysis

The cellulose purity enables its utilization as dissolving grade cellulose (Dongfang et al., 2012). This includes applications in many industries such as textile, pharmaceutical, food

Viscose cellulose. The purity of the cellulose enable it to be used as viscose cellulose for several applications. In some cases the cellulose can be further processed to improve the properties. For example, it can be bleached to improve the brightness.

Viscose Fiber. Man-made biodegradable fibers of rayon that are spun from viscose pulp and have application in apparels, home textile, dress material, and knitted wear as well as in non-woven applications.

Because of the high purity obtained after the treatment, the mild conditions and the characteristic of the solvent, the cellulose can be used in advance applications such as the production of nanocellulose cellulose (Klemm et al., 2009).

Other applications that require a high purity cellulose are in the scope of the applications

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure from 1 to 10 should be construed as supporting a range from 2 to 8, from 3 to 7, 5, 6, from 1 to 9, from 3.6 to 4.6, from 3.5 to 9.9, and so forth.

All references to singular characteristics or limitations shall include the corresponding plural characteristic or limitation, and vice versa, unless otherwise specified or clearly implied to the contrary by the context in which the reference is made.

The processes described herein can be run in batch mode, semi-continuous mode, and/or continuous mode, all of which are explicitly included herein.

All combinations of method or process steps as used herein can be performed in any order, unless otherwise specified or clearly implied to the contrary by the context in which the referenced combination is made.

The methods described and claimed herein can comprise, consist of, or consist essentially of the essential elements and limitations of the disclosed methods, as well as any additional or optional ingredients, components, or limitations described herein or otherwise useful in synthetic organic chemistry.

### Example 1

750 g of wet (30% moisture) white birch wood chips were placed in a metal basket with a 1 mm diameter screen. A re-circulated solvent comprising 80/20 GVL/water by weight, 0.1 M sulfuric acid, and soluble material from the white birch was used to treat the white birch at 140° C. for 90 minutes. The amount of lignin, hemicellulose and cellulose solubilized increased during the first 45 minutes. After that time only the cellulose solubilization increased. After 90 minutes the liquid was partially separated from the solid by removing it from the reactor. The cellulose remaining in the reactor was washed with a combination of water and GVL 3 times and then with hot water to recover the solvent remaining in the cellulose. 37.5% of the initial white birch remained as solids. Of the solids, 91.5% were cellulose and the rest identified as lignin, hemicellulose, and other impurities. GVL was detected in the solids.

### Example 2

The solids from example 1 were bleached following a DEPDD sequence. The alpha cellulose content of the solids after bleaching was 74.5% with cellulose purity >95%. The viscosity of the sample was 3cP according with Tappi/Ansi T 230 m 08.

### Example 3

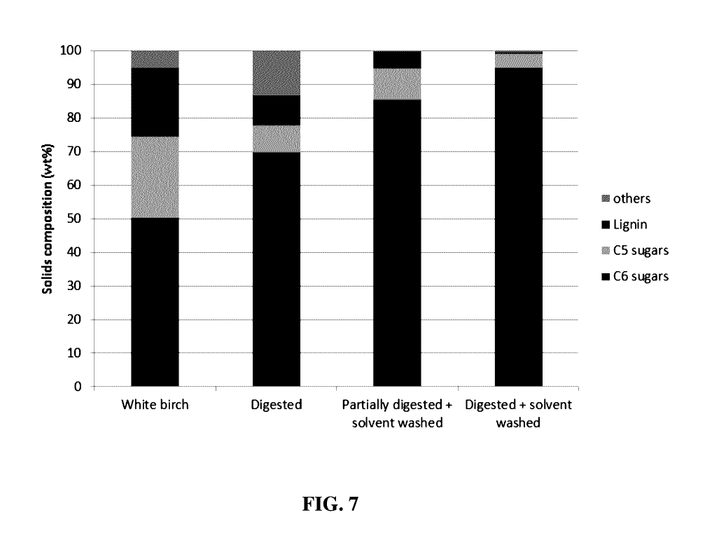

The solids from example 1 and example 2 were re-dispersed in water and used to make a paper sheet. The prepared paper sheets were subjected to refinement using 100 to 1000 revolutions. The pulp was analyzed for properties such as basis weight, caliper, bulk/density, burst, tear, consistency, and tensile index. For 100 to 1000 rev. refining, GVL pulp demonstrated caliper of 0.43 mm to 0.34 mm, an apparent density of 0.97 to 0.76 g/cm3, burst index of 2.0 to 2.8 kPa*m2/g, tear index of 3.5 to 2.6 mN*m2/g, tensile index of 36.0 to 55.5 Nm/g, strain at rupture of 2.6 to 2.8%, tensile energy adsorbed at rupture of 40.2 to 64.0 J/m2 and elastic modulus of 290 to 344 kN/m. The strength versus refining and strength versus density plots are presented in FIG. 7.

### Example 4

5 g white birch wood (5% moisture) were placed in a 60 ml glass reactor. A solvent comprising 70/30 GVL/water by weight, 0.1 M sulfuric acid, and soluble material from the white birch was used to treat the white birch at 125° C. for 180 minutes. After 180 minutes the liquid was partially separated from the solid. The cellulose was washed with a combination of water and GVL 3 times and then with hot water to recover the solvent remaining in the cellulose. 40.8% of the initial white birch remained as solids. Of the solids, 94.0% were cellulose and the rest identified as lignin, hemicellulose, and other impurities. GVL was detected in the solids.

### Example 5

5 g white birch wood (5% moisture) was placed in a 60 ml glass reactor. A solvent comprising 70/30 GVL/water by weight, 0.1 M sulfuric acid, and soluble material from the white birch was used to treat the white birch at 125° C. for 180 minutes. After 180 minutes the liquid was partially separated from the solid. The cellulose was washed with hot water to recover the solvent remaining in the cellulose. 42.8% of the initial white birch remained as solids. Of the solids, 89.5% were cellulose and the rest identified as lignin, hemicellulose, and other impurities. GVL was detected in the solids.

### Example 6

5 g white birch wood (5% moisture) was placed in a 60 ml glass reactor. The liquid used to wash the cellulose in the example 4 was used as solvent after the addition of 0.05 M sulfuric acid to treat the white birch at 125° C. for 180 minutes. After 180 minutes the liquid was partially separated from the solid. The cellulose was washed with a combination of water and GVL three times and then with hot water to recover the solvent remaining in the cellulose. 40.8% of the initial white birch remained as solids.

### Example 7

2 kg of white birch wood chips (1×1×¼ in) were introduced in a twin digester reactor into a basket with 1 mm diameter pores and treated with recirculating GVL/water solvent (70/30 w/w) and 0.1% H₂SO₄ at 125° C. for 3 hours such that the solid/liquid ratio is 6:1. After the reaction the pulp was washed with 70/30 GVL/water once and another two times with 50/50 mixture of GVL/water. Subsequently, the pulp was washed three more times with water. The pulp yield out of the reactor was 48%, while the screened pulp yield was 42%. The screened pulp had a kappa number of 20.

### Example 8

The pulp from example 7 was bleached using a bleaching process to increase the brightness without decreasing the viscosity (DED). The viscosity of the bleached pulp was 15.08 cps

### Example 9

The bleached pulp from example 8 was analyzed for viscose pulp properties. The alpha cellulose content measured using Tappi 203 method was 91.2%, the beta cellulose was 4.9%, the hemicellulose (gamma cellulose) was 3.9%, the pentosans measured using NREL/TP-510-42618 structural carbohydrate analysis was 3.1%, Tthe kappa number, ash content and acid insoluble content was too small to be determined or zero. The high alpha cellulose content, low hemicellulose, minimal or absence of ash, acid insoluble and lignin are advantageous for viscose pulp and fiber production.

### Example 10

Several batches of empty fruit brunches were shredded and added at a 10 wt % biomass loading to a solvent comprising 80/20 GVL water by weight and 0.075 M sulfuric acid in 10 mL glass reactors. The glass reactors were heated at 130° C. for different times. At all the times, more than 90% of the hemicellulose was removed from the solids. The amount of cellulose solubilized increased with time. After 30 and 45 minutes more than 80% of the hemicellulose dissolved is present as soluble C₅ sugar monomer or oligomers. After 45 minutes more than 95% of the hemicellulose is present as soluble C₅ sugars or oligomers or as furfural.

### Example 11

1 kg of wet empty fruit bunches were treated with 6.5 kg of solvent comprised by 80/20 GVL water by weight and 0.1 M sulfuric acid. The wet empty fruit bunches were placed in a metal basket with a 1 mm diameter screen and the liquid was re-circulated for 60 min at 140° C. 466 g of solids were recovered after the reaction. The cellulose content of the solids was 72% indicating that a correct washing of the cellulose is necessary to produce cellulose with high purity. The alpha cellulose content of the solids was 75.5%. The solids can be bleached to increase the purity to 85.5%. Further treatment with NaOH can increase the purity of the solids to 99.4%. cls IV. REFERENCES

The following references, to the extent that they provide exemplary procedural or other details supplementary to those set forth herein, are specifically incorporated herein by reference.
Alonso et al., Energy Environ. Sci. 6:76-80, 2012.
Button et al. The Word of Market Pulp—WOMP LLC, 2003.
Dhepe & Fukuoka, Chemsuschem 1:969-975, 2008.
Dongfang et al., in Functional Materials from Renewable Sources 1107:167-189, American Chemical Society, 2012.
Fang and Sixta, Chem Sus Chem 8:73-76, 2015.
Gallo et al. Green Chem. 15:85-90, 2013.
Gallo et al., Top. Catal. 56:1775-1781, 2013.
Gürbüz et al., Angew. Chem. Int. Ed. 52:1270-1274, 2013.
Klemm et al., Macromol. Symp. 280:60-71, 2009.
Lee and Jeffries, Bioresour. Technol. 102:5884-5890, 2011.
Mellmer et al., Angew. Chem. Int. Ed. 53:11872-11875, 2014b.
Mellmer et al., Green Chem. 16:4659-4662, 2014a.
Mittal et al., Green Chem. 17:1546-1558, 2015.
Nguyen et al., Chem Sus Chem 8:1716-1725, 2015.
Nissan, Tappi J. 67:153-153, 1984.
Van de Vyver et al., Chemcatchem 3:82-94, 2011.
Wettstein et al., Energy Environ. Sci. 5:8199, 2012.
Wiley: Aqueous Pretreatment of Plant Biomass for Biological and Chemical Conversion to Fuels and Chemicals—Charles E. Wyman. at <world-wide-web at wiley.com/WileyCDA/WileyTitle/productCd-0470972025.html>
Xu and Huang, Appl. Biochem. Biotechnol. 174:43-62, 2014.
Zakzeski et al., Chem. Rev. 110:3552-3599, 2010.
Zhu et al., Process Biochem. 47:785-791, 2012.

1-87. (canceled)

88. A method to fractionate lignocellulosic biomass comprising:
(a) providing one or more sources of solid cellulose;
(b) treating said source of cellulose with water, an acid and an aprotic solvent that preferentially solubilize the lignocellulosic biomass other than cellulose;
(c) generating a liquid stream of the materials in step (b);
(d) separating the solid cellulose from the liquid stream of step (c) at the fractionation temperature or higher;
(e) washing the cellulose of step (d) with a mixture of at least water and an organic solvent at the fractionation temperature or higher to prevent the re-precipitation of dissolved material remaining in the liquid; and
(f) treating the solid cellulose to recover the organic solvents.

89. The method of claim 88, wherein separation and washing steps are performed at less than 20° C. lower than the fractionation temperature.

90. The method of claim 88, wherein lignin and other water-insoluble degradation products are removed from the solvent by precipitation by adding water.

91. The method of claim 90, wherein the precipitation of the lignin and other water-insoluble materials takes place at the fractionation temperature.

92. The method of claim 91, wherein the precipitation of the lignin and other water-insoluble materials takes place at less than 20° C. difference than the fractionation temperature.

93. The method of claim 88, wherein the sugars solubilized in the liquid stream are converted into furfural.

94. The method of claim 88, wherein the sugars solubilized in the liquid stream are converted into furfural after separating the lignin and other water-insoluble products.

95. The method of claim 94, wherein at least part of the water is removed by evaporating before the production of furfural.

96. The method of claim 88, wherein said aprotic solvent is GVL, THF, a lactone, a lactam, a furan, a pyran, a sulfone, an ether or an ester.

97. The method of claim 88, wherein >80% of the dissolved hemicellulose C5 sugars are retained as soluble carbohydrates and more than 95% of the dissolved hemicellulose C5 sugars are retained as soluble carbohydrate or furfural.

98. The method of claim 88, wherein the solid/liquid ratio is above 0.15.

99. The method of claim 88, wherein less than 15% of the cellulose is solubilized.

100. The method of claim 88, further comprising treating the solid cellulose to increase the cellulose purity above 98%.

101. The method of claim 88, wherein separation comprises filtration, centrifugation, decantation, or compression.

102. The method of claim 88, wherein the accessible surface of the cellulose is increased.

103. The method of claim 88, wherein the obtained cellulose exhibits a pentosane content of less than 4%, less than 3% or less than 2%.

104. The method of claim 88, wherein the obtained cellulose exhibits a kappa number of less than 1, less than 0.5, less than 0.3, less than 0.2 or less than 0.1.

105. A method of producing a nano-crystalline cellulose composition comprising:
(a) providing one or more sources of solid cellulose;
(b) treating said source of cellulose with water, an acid and an aprotic solvent that preferentially solubilize lignocellulosic biomass other than cellulose;
(c) generating a liquid stream of the materials in step (b);
(d) separating the solid cellulose from the liquid stream of step © at the fractionation temperature or higher;
(e) washing the cellulose of step (d) with a mixture of at least water and an organic solvent at the fractionation temperature or higher to prevent the re-precipitation of dissolved material remaining in the liquid;
(f) treating the solid cellulose to recover the organic solvents; and
(g) separating solid nano-crystalline cellulose from the liquid stream of step (f).

106. The method of claim 105, wherein further comprising purifying cellulose before producing nano-crystalline cellulose.

107. The method of claim 105, wherein the cellulose is treated with concentrated acid to remove the non-crystalline cellulose prior to separating the nano-crystalline cellulose and wherein at least a portion of the non-nano crystalline cellulosic portion of the cellulose is further treated to produce glucose, HMF, levulinic acid, GVL or a derivative thereof.
